(19) United States
(12) Patent Application Publication
(10) Pub. No.: US 2014/0141089 A1
(43) Pub. Date: May 22, 2014

(54) Nanoparticles, Compositions Thereof, and Methods of Use, and Methods of Making the Same

(71) Applicant: Hongjun Liang, Arvada, CO (US)

(72) Inventor: Hongjun Liang, Arvada, CO (US)

(73) Assignee: Colorado School of Mines, Golden, CO (US)

(21) Appl. No.: 12/704,416

(22) Filed: Feb. 11, 2010

Related U.S. Application Data
(60) Provisional application No. 61/151,725, filed on Feb. 11, 2009.

(51) Int. Cl. A61K 9/14; A61K 31/4745; A61K 31/573; A61K 31/5415; A61K 31/5513; A61P 35/00; A61P 29/00; A61P 31/10; A61P 25/18; A61P 7/02; A61K 39/395; A61P 27/02; A61P 25/22; A61P 9/12; A61P 9/06; A61P 25/08; A61P 3/10; A61P 9/10; A61P 3/06; A61P 3/04; A61P 15/10; A61P 25/16; A61P 31/04; A61P 25/28; A61P 31/12; A61P 25/24; A61K 31/635; A61K 39/00; A61K 31/337

(52) U.S. Cl. 424/491; 424/498; 514/449; 514/283; 514/180; 514/223.2; 514/221; 424/130.1; 514/158; 424/184.1

(57) ABSTRACT

The disclosure is directed to a nanoparticle comprising a porous framework core including a porous framework material and a compound, and a lipid layer disposed on the surface of the porous framework core.

### CROSS REFERENCE TO RELATED APPLICATIONS

The present application claims benefit of priority under 35 U.S.C. §119(e) to U.S. provisional application 61/151,725 filed Feb. 11, 2009, the contents of which are hereby incorporated by reference in its entirety. The present application is related to U.S. provisional application 61/297,533, filed Jan. 22, 2010, titled HARVESTING MICRO ALGAE.

### FIELD

The present disclosure relates to nanoparticles useful as diagnostic and therapeutic compound delivery systems, methods of treating and diagnosing diseases and disorders using the nanoparticles, and methods of making the same.

### BACKGROUND

Delivery of therapeutic and diagnostic compounds often involves crossing biological barriers to reach target sites. Delivery of these therapeutic and diagnostic compounds often requires surgically implanting devices for administering the therapeutic or diagnostic compound. Alternatively, intravenous delivery involves conjugation or chemical bonding of the therapeutic or diagnostic compound to a carrier molecule. While intravenous delivery of therapeutic or diagnostic compounds may be more versatile and efficient than surgical implantation, it is often costly and/or inefficient to prepare the compounds.

Several aspects of delivering therapeutic and diagnostic agents to patients may be desirable. These include the ability to target specific cells or tissues (K. E. Uhrich, et al., Chem. Rev., 99, 3181 (November, 1999), T. M. Allen, Nat. Rev. Cancer, 2, 750 (October, 2002)); the ability to deliver therapeutic agents within a defined time frame; the ability to overcome biological barriers which may degrade, alter, or clear the agents from the body (R. K. Jain, Nat. Med. 4, 655 (June, 1998), M. Ferrari, Nat. Rev. Cancer 5, 161 (March, 2005)); the ability to sequester toxic therapeutic compounds; and the capacity to shuttle a wide variety of therapeutic compounds with different physical characteristics.

Some existing delivery techniques that rely on derivatization of the therapeutic or diagnostic compound, or covalently linking the agent to a delivery molecule tend to be costly and inefficient. Liposome-based delivery meets only a few of the desirable aspects of drug delivery. In many circumstances, liposomes are incompatible with low-solubility therapeutic and diagnostic compounds, which account for nearly 70% of early pre-clinical development of therapeutic compounds (M. E. Napier, J. M. Desimone, Polym. Rev. 47, 321 (2007)).

The present disclosure has been developed against this backdrop.

### SUMMARY

In one aspect, the present disclosure is directed to a nanoparticle comprising a porous framework core including a porous framework material and a compound, and a lipid layer disposed on the surface of the porous framework core. The porous framework material may include one or more metal oxides or non-metal oxides. The porous framework material may include a silane. The lipid layer may further include polymers, proteins, peptides, or other molecules contained within the membrane.

The compounds may be therapeutic or diagnostic agents. The present disclosure is further directed to a pharmaceutical composition comprising a nanoparticle and a pharmaceutically acceptable vehicle. The nanoparticles or pharmaceutical compositions may be used to diagnose or treat a disease or disorder. The compounds may be compounds that are difficult to administer, such as toxic compounds, hydrophobic or amphipathic compounds, or compounds that are ineffective by traditional methods of administration unless administered in large dosages.

### BRIEF DESCRIPTION OF THE DRAWINGS

FIG. 1 is a diagram of a nanoparticle showing a lipid bilayer disposed on a porous framework core.

FIG. 2 shows various types of porous framework material shapes and sizes.

FIG. 3 is a diagram showing molecules, polymers, and proteins associated with the lipid bilayer of a nanoparticle.

### DETAILED DESCRIPTION

A dash (“-”) that is not between two letters or symbols is used to indicate a point of attachment for a moiety or substituent. For example, —CONH₂ is attached through the carbon atom.

“Alkyl” by itself or as part of another substituent refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In certain embodiments, an alkyl group comprises from 1 to 20 carbon atoms (C₁₋₂₀), in certain embodiments, from 1 to 10 carbon atoms (C₁₋₁₀), from 1 to 8 carbon atoms (C₁₋₈), from 1 to 6 carbon atoms (C₁₋₆), from 1 to 4 carbon atoms (C₁₋₄), and in certain embodiments, from 1 to 3 carbon atoms (C₁₋₃).

“Alkanyl” by itself or as part of another substituent refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl or heteroarylalkyl as defined herein. Representative examples include, but are not limited to formyl, acetyl, cyclohexylcarbonyl, cyclohexylmethylcarbonyl, benzoyl, benzylcarbonyl and the like.

“Acylamino” by itself or as part of another substituent refers to a radical —NR³¹C(O)R³², where R³¹ and R³² are independently hydrogen, alkyl, cycloalkyl, cycloheteroalkyl, aryl, arylalkyl, heteroalkyl, heteroaryl or heteroarylalkyl as defined herein. Representative examples include, but are not limited to formamido, acetamido and benzamido.

“Acyloxy” by itself or as part of another substituent refers to a radical —OC(O)R³³, where R³³ is alkyl, cycloalkyl, cycloheteroalkyl, aryl, arylalkyl, heteroalkyl, heteroaryl or heteroarylalkyl as defined herein. Representative examples include, but are not limited to acetoxy, isobutyroyloxy, benzoyloxy, phenylacetoxy and the like.

“Alkoxy” by itself or as part of another substituent refers to a radical —OR³⁴ where R³⁴ represents an alkyl or cycloalkyl group as defined herein. Representative examples include, but are not limited to, methoxy, ethoxy, propoxy, butoxy, cyclohexyloxy and the like.

“Alkylamino” means a radical —NHR where R represents an alkyl or cycloalkyl group as defined herein. Representative examples include, but are not limited to, methylamino, ethylamino, 1-methylethylamino, cyclohexyl amino and the like.

“Alkoxycarbonyl” by itself or as part of another substituent refers to a radical —C(O)—OR³⁵ where R³⁵ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Alkoxycarbonylamino” by itself or as part of another substituent refers to a radical —NR³⁶C(O)—OR³⁷ where R³⁶ represents an alkyl or cycloalkyl group and R³⁷ is alkyl, cycloalkyl, cycloheteroalkyl, aryl, arylalkyl, heteroalkyl, heteroaryl, heteroarylalkyl as defined herein. Representative examples include, but are not limited to, methoxycarbonylamino, tert-butoxycarbonylamino and benzyloxycarbonylamino.

“Alkoxycarbonyloxy” by itself or as part of another substituent refers to a radical —OC(O)—OR³⁸ where R³⁸ represents an alkyl or cycloalkyl group as defined herein. Representative examples include, but are not limited to, methoxycarbonyloxy, ethoxycarbonyloxy and cyclohexyloxycarbonyloxy.

“Alkylsulfinyl” refers to a radical —S(O)R where R is an alkyl or cycloalkyl group as defined herein. Representative examples include, but are not limited to, methylsulfinyl, ethylsulfinyl, propylsulfinyl, butylsulfinyl and the like.

“Alkylsulfonyl” refers to a radical —S(O)₂R where R is an alkyl or cycloalkyl group as defined herein. Representative examples include, but are not limited to, methylsulfonyl, ethylsulfonyl, propylsulfonyl, butylsulfonyl and the like.

“Alkylthio” refers to a radical —SR where R is an alkyl or cycloalkyl group as defined herein that may be optionally substituted as defined herein. Representative examples include, but are not limited to methylthio, ethylthio, propylthio, butylthio and the like.

“Aryl” by itself or as part of another substituent refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryl group is from 6 to 20 carbon atoms. In other embodiments, an aryl group is from 6 to 12 carbon atoms.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In some embodiments, an arylalkyl group is (C₆-C₃₀) arylalkyl, e.g., the alkanyl, alkenyl or alkynyl moiety of the arylalkyl group is (C₁-C₁₀) and the aryl moiety is (C₆-C₂₀). In other embodiments, an arylalkyl group is (C₆-C₂₀) arylalkyl, e.g., the alkanyl, alkenyl or alkynyl moiety of the arylalkyl group is (C₁-C₈) and the aryl moiety is (C₆-C₁₂).

“Aryloxycarbonyl” refers to a radical —C(O)—O-aryl where aryl is as defined herein.

“Aryloxy” refers to a radical —C—O-aryl where aryl is as defined herein.

“Carbamoyl” by itself or as part of another substituent refers to the radical —C(O)NR³⁹R⁴⁰ where R³⁹ and R⁴⁰ are independently hydrogen, alkyl, cycloalkyl or aryl as defined herein.

“Carbamoyloxy” by itself or as part of another substituent refers to the radical —OC(O)NR⁴¹R⁴² where R⁴¹ and R⁴² are independently hydrogen, alkyl, cycloalkyl or aryl as defined herein.

“Pharmaceutically acceptable salt” refers to a salt of a compound, which possesses the desired pharmacological activity of the parent compound. Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like. In certain embodiments, a pharmaceutically acceptable salt is the hydrochloride salt. In certain embodiments, a pharmaceutically acceptable salt is the sodium salt.

“Salt” refers to a salt of a compound, including, but not limited to, pharmaceutically acceptable salts.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to a compound as described herein and at least one pharmaceutically acceptable vehicle, with which the described compound is administered to a patient.

“Solvate” refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

“Conjugate acid of an organic base” refers to the protonated form of a primary, secondary or tertiary amine or heteroaromatic nitrogen base. Representative examples include, but are not limited to, triethylammonium, morpholinium and pyridinium.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In some embodiments, the cycloalkyl group is (C₃-C₁₀) cycloalkyl. In other embodiments, the cycloalkyl group is (C₃-C₇) cycloalkyl.

“Cycloheteroalkyl” by itself or as part of another substituent refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine, and the like.

“Dialkylamino” by itself or as part of another substituent refers to the radical —NR⁴³R⁴⁴ where R⁴³ and R⁴⁴ are independently alkyl, cycloalkyl, cycloheteroalkyl, arylalkyl, heteroalkyl or heteroarylalkyl, or optionally R⁴³ and R⁴⁴ together with the nitrogen to which they are attached form a cycloheteroalkyl ring.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —O—O—, —S—S—, —O—S—, —NR⁴⁵R⁴⁶, —═N—N═—, —N═N—, —N═N—NR⁴⁷R⁴⁸, —PR⁴⁹—, —P(O)₂—, —POR⁵⁰—, —O—P(O)₂—, —SO—, —SO₂—, —SnR⁵¹R⁵²— and the like, where R⁴⁵, R⁴⁶, R⁴⁷, R⁴⁸, R⁴⁹, R⁵⁰, R⁵¹ and R⁵² are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, □-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is from 5-20 membered heteroaryl, more preferably from 5-10 membered heteroaryl. Certain heteroaryl groups are those derived from thiophene, pyrrole, benzothiophene, benzofuran, indole, pyridine, quinoline, imidazole, oxazole and pyrazine

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In some embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In other embodiments, the heteroarylalkyl group is a 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Sulfonamido” by itself or as part of another substituent refers to a radical —NR⁵³S(O)₂R⁵⁴, where R⁵³ is alkyl, substituted alkyl, cycloalkyl, cycloheteroalkyl, aryl, substituted aryl, arylalkyl, heteroalkyl, heteroaryl or heteroarylalkyl and R⁵⁴ is hydrogen, alkyl, cycloalkyl, cycloheteroalkyl, aryl, arylalkyl, heteroalkyl, heteroaryl or heteroarylalkyl as defined herein. Representative examples include, but are not limited to methanesulfonamido, benzenesulfonamido and p-toluenesulfonamido.

“Aromatic Ring System” by itself or as part of another substituent refers to an unsaturated cyclic or polycyclic ring system radical having a conjugated π electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System” by itself or as part of another substituent refers to a aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Halo” means fluoro, chloro, bromo, or iodo radical.

“Heteroalkyloxy” means an —O-heteroalkyl where heteroalkyl is as defined herein.

“Heteroaryloxycarbonyl” refers to a radical —C(O)—OR where R is heteroaryl as defined herein.

Substituted” or “substituent” refers to a group in which one or more hydrogen atoms are each independently replaced with the same or different substituent(s). Typical substituents include, but are not limited to, —X, —R²⁹, —O, ═O, —OR²⁹, —SR²⁹, —S⁻, ═S, —NR²⁹R³⁰, ═NR²⁹, —CX₃, —CF₃, —CN, —OCN, —SCN, —NO, —NO₂, ═N₂, —N₃, —S(O)₂O⁻, —S(O)₂OH, —S(O)₂R²⁹, —OS(O₂)O⁻, —OS(O)₂R²⁹, —P(O)(O⁻)₂, —P(O)(OR²⁹)(O⁻), —OP(O)(OR²⁹)(OR³⁰), —C(O)R²⁹, —C(S)R²⁹, —C(O)OR²⁹, —C(O)NR²⁹R³⁰, —C(O)O⁻, —C(S)OR²⁹, —NR³¹C(O)NR²⁹R³⁰, —NR³¹C(S)NR²⁹R³⁰, —NR³¹C(NR²⁹)NR²⁹R³⁰ and —C(NR²⁹)NR²⁹R³⁰, where each X is independently a halogen; each R²⁹ and R³⁰ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR³¹R³², —C(O)R³¹ or —S(O)₂R³¹ or optionally R²⁹ and R³⁰ together with the atom to which they are both attached form a cycloheteroalkyl or substituted cycloheteroalkyl ring; and R³¹ and R³² are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Sulfonic acids derivatives” as used herein are a class of organic acid radicals with the general formula RSO₃H or RSO₃. An oxygen, suflur, or R moiety can serve as a point of attachment. Sulfonic acid salt derivatives substitute a cationic salt (e.g. Na⁺, K⁺, etc.) for the hydrogen on the sulfate group. In various embodiments, the deprotonated sulfonic acid group can be used as the point of attachment to a therapeutic or targeting group, optionally via a linker. Examples of sulfonic acid derivatives and include, but are not limited to, 2-methyl-2-propane-1-sulfonic acid-sodium salt, 2-sulfoethyl methacrylate, 3-phenyl-1-propene-2-sulfonic acid-p-toluidine salt, 3-sulfopropyl acrylate-potassium salt, 3-sulfopropyl methacrylate-potassium salt, ammonium 2-sulfatoethyl methacrylate, styrene sulfonic acid, 4-sodium styrene sulfonate.

“Anhydride derivatives” as used herein refer to a compound or radical having the chemical structure R₁C(O)OC(O)R₂. The carboxyl groups, optionally after removal of R₁ or R₂ groups, can be used as the point of attachment to a therapeutic or targeting group, optionally via a linker. Examples of anhydride derivatives include, but are not limited to, acrylic anhydride, methacrylic anhydride, maleic anhydride, and 4-methacryloxyethyl trimellitic anhydride

“Hydroxyl derivative” as used herein refers to a compound or radical having the structure ROH. The deprotonated hydroxyl group can be used as the point of attachment to a therapeutic or targeting group, optionally via a linker. Example of hydroxyl derivatives include, but are not limited to, vinyl alcohol, 2-hydroxyethyl acrylate, 2-hydroxyethyl methacrylate, 2-allyl-2-methoxyphenol, divinyl glycol, glycerol monomethacrylate, poly(propylene glycol) monomethacrylate, N-(2-hydroxypropyl)methacrylamide, hydroxymethyldiacetoneacrylamide, poly(ethylene glycol) monomethacrylate, N-methacryloylglycylglycine, N-methacryloylglycyl-DL-phenylalanylleucylglycine, 4-methacryloxy-2-hydroxybenzophenone, 1,1,1-trimethylolpropane diallyl ether, 4-allyl-2-methoxyphenol, hydroxymethyldiacetoneacrylamide, N-methylolacrylamide, and sugar based monomers.

“Amine derivatives” are compound or radicals thereof having a functional group containing at least one nitrogen, and having the structure RNR′R″. R, R′ and R″ in amine derivatives can each independently be any desired substituent, including but not limited to hydrogen, halides, and substituted or unsubstituted alkyl, alkoxy, aryl or acyl groups. “Amide derivatives” as used herein refer to compounds having the structure RC(O)NR′R″. The R, R′ and R″ in amide derivatives can each independently be any desired substituent, including but not limited to hydrogen, halides, and substituted or unsubstituted alkyl, alkoxy, aryl or acyl groups. The amine or amide group can be used as the point of attachment to a therapeutic or targeting group, optionally via a linker. Examples of amines and amides include, but are not limited to, 2-(N,N-diethylamino)ethyl methacrylate, 2-(N,N-diethylamino)ethyl acrylate, N-[2-(N,N-dimethylamino)ethyl]methacrylamide, N-[3-(N,N-dimethylamino)propyl]acrylamide, diallylamine, methacryloyl-L-lysine, 2-(tert-butylamino)ethyl methacrylate, N-(3-aminopropyl)methacrylamide hydrochloride, 3-dimethylaminoneopentyl acrylate, N-(2-hydroxypropyl)methacrylamide, N-methacryloyl tyrosine amide, 2-diisopropylaminoethyl methacrylate, 3-dimethylaminoneopentyl acrylate, 2-aminoethyl methacrylate hydrochloride, hydroxymethyldiacetoneacrylamide, N-(iso-butoxymethyl)methacrylamide and N-methylolacrylamide.

“Silane derivative” as used herein refers to compounds or radicals thereof having at least one substituent having the structure RSiR′R″R′″. R, R′ and R″ can each independently be any desired substituent, including but not limited to hydrogen, alkyl, alkoxy, aryl or acyl groups. The silane group can be used as the point of attachment to a therapeutic or targeting group, optionally via a linker. Examples of silane derivatives include, but are not limited to, 3-methacryloxypropyl trimethoxysilane, vinyltriethoxysilane, 2-(trimethylsiloxy)ethyl methacrylate, 1-(2-trimethylsiloxyethoxy)-1-trimethylsiloxy-2-methylpropene

“Phosphate derivatives” as used herein refer to compounds or radicals thereof having at least one compound containing the structure RR′R″PO₄. R, R′ and R″ can each independently be any desired substituent, including but not limited to hydrogen, alkyl, alkoxy, aryl or acyl groups. The phosphate group can be used as the point of attachment to a therapeutic or targeting group, optionally via a linker. Examples of phosphate derivatives include, but are not limited to, monoacryloxyethyl phosphate and bis(2-methacryloxyethyl) phosphate.

“Nitro derivatives” as used herein refer to compounds or radicals thereof having an NO₂ group. The nitro group can be used as the point of attachment to a therapeutic or targeting group, optionally via a linker. Examples include, but are not limited to, o-nitrobenzyl methacrylate, methacryloylglycyl-DL-phenylalanyl-L-leucyl-glycine 4-nitrophenyl ester, methacryloylglycyl-L-phenylalanyl-L-leucyl-glycine 4-nitrophenyl ester, N-methacryloylglycylglycine 4-nitrophenyl ester, 4-nitrostyrene

“Succinimide derivative” as used herein refers to compounds or radicals thereof having the group

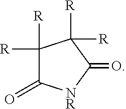

The succinyl R groups can be substituted by any substituent, for example and substituted or unsubstituted alkyl, alcoxy, aryl groups. Typically, the succinimide group is attached to a compound via a covalent bond at the nitrogen. The succinimide group can be used as the point of attachment to a therapeutic or targeting group, optionally via a linker. A succinimide derivative can be a sulfo-containing succinimide derivative. N-acryloxysuccinimide is an exemplary succinimide derivative.

“Halide derivatives” as used herein refer to compounds or radicals thereof having a halide substituent. The halide group can be used as the point of attachment to a therapeutic or targeting group, optionally via a linker. Examples include, but are not limited to, vinyl chloride, 3-chlorostyrene, 2,4,6-tribromophenyl acrylate, 4-chlorophenyl acrylate, 2-bromoethyl acrylate. Non-limiting examples include, but are not limited to, divinylbenzene, ethylene glycol diacrylate, N,N-diallylacrylamide, and allyl methacrylate.

“Morpholine derivatives” as used herein refer to compounds or radicals thereof having the structure:

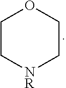

Typically, the amine group serves as the point of attachment to other compounds. The morpholine group can be used as the point of attachment to a therapeutic or targeting group, optionally via a linker. Examples of morpholine derivatives include, but are not limited to, N-acryloylmorpholine, 2-N-morpholinoethyl acrylate and 2-N-morpholinoethyl methacrylate.

“Cyano derivatives” as used herein refer to compounds or radicals thereof having the structure RCN. R can each independently be any desired substituent. The cyano group can be used as the point of attachment to a therapeutic or targeting group, optionally via a linker. Examples of cyano derivatives include, but are not limited to, 2-cyanoethyl acrylate.

“Epoxide derivatives” as used herein refer to compounds or radicals thereof having the following chemical structure:

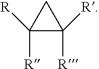

R, R′, R″, and R′″ can each independently be any desired substituent. The epoxide group can be used as the point of attachment to a therapeutic or targeting group, optionally via a linker. Examples of epoxide derivatives include, but are not limited to, glycidyl methacrylate.

“Ester derivatives” as used herein refer to a compound or a radical thereof having the generic chemical structure RC(O)OR′. R and R′ can each independently be any desired substituent. The ester group can be used as the point of attachment to a therapeutic or targeting group, optionally via a linker. Examples include, but are not limited to, methyl acrylate, methyl methacrylate, tert-butyl acrylate, tert-butyl methacrylate, vinyl acetate, benzyl acrylate and benzyl methacrylate.

“Ether derivatives” as used herein refer to a compound or a radical thereof having the generic chemical structure R—O—R′. The ether group can be used as the point of attachment to a therapeutic or targeting group, optionally via a linker. Examples include, but are not limited to, methyl vinyl ether, butyl vinyl ether, 2-chloroethyl vinyl ether, cyclohexyl vinyl ether.

“Carbazole derivatives” as used to herein refer to a compound or radical thereof having the structure

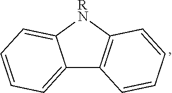

and any substitutions at any site thereof. The carbazole group can be used as the point of attachment to a therapeutic or targeting group, optionally via a linker. Examples of carbazole derivatives include but are not limited to, N-vinylcarbazole.

“Azide derivatives” as used herein refer to a compound or a radical thereof having the structure N═N═N. The azide group can be used as the point of attachment to a therapeutic or targeting group, optionally via a linker. Examples of azide derivatives include, but are not limited to, 2-hydroxy-3-azidopropyl methacrylate, 2-hydroxy-3-azidopropyl acrylate, 3-azidopropyl methacrylate.

The term “maleimide derivative” as referred to herein refers to a compound or a radical thereof having the structure:

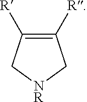

R, R′ and R″ can each independently be any desired substituent.

The term “thiolate” refers to a compound or radical thereof having a —SR structure, where R can be any desired substituent.

The term “thioether” refers to a compound or radical thereof having the structure R—S—CO—R′, where R and R′ can each independently be any desired substituent.

The term “thioester” refers to a compound or radical thereof having the structure R—S—CO—R′, where R and R′ can each independently be any desired substituent.

The term “carboxylate” refers to a compound or radical thereof having the structure RCOO—, where R can be any desired substitutent.

The term “phosphonate” refers to a compound or radical thereof having the structure R—PO(OH)₂ or R—PO(OR′)₂ where R and R′ can each independently be any desired substituent.

The term “phosphinate” refers to a compound or radical thereof having the structure OP(OR)R′R″ where R, R′ and R′ can each independently be any desired substituent.

The term “sulphonate” refers to a compound or radical thereof having the structure RSO₂O⁻ where R can be any desired substituent.

The term “sulphate” refers to a compound or radical thereof having the structure RSO₄ where R can be any desired substituent.

A “reducing agent” is an element or a compound that reduces another species. Exemplary reducing agents include, but are not limited to, ferrous ion, lithium aluminium hydride (LAIN, potassium ferricyanide (K₃Fe(CN)₆), sodium borohydride (NaBH₄), sulfites, hydrazine, diisobutylaluminum hydride (DIBAH), primary amines, and oxalic acid (C₂H₂O₄).

“Treating” or “treatment” of any disease or disorder refers, in some embodiments, to ameliorating the disease or disorder (i.e., arresting or reducing the development of the disease or at least one of the clinical symptoms thereof). In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter), or both. In still other embodiments, “treating” or “treatment” refers to delaying the onset of the disease or disorder.

The term “antibody” refers to a monomeric or multimeric protein comprising one or more polypeptide chains that binds specifically to an antigen. An antibody can be a full length antibody or an antibody fragment.

By “full length antibody,” herein is meant the structure that constitutes the natural biological form of an antibody, including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, CH1 (Cγ1), CH2 (Cγ2), and CH3 (Cγ3). In some mammals, for example in camels and llamas, IgG antibodies may consist of only two heavy chains, each heavy chain comprising a variable domain attached to the Fc region.

“Antibody fragments” are portions of full length antibodies that bind antigens. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883), (viii) bispecific single chain Fv dimers (PCT/US92/09965) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448). In certain embodiments, antibodies are produced by recombinant DNA techniques. Other examples of antibody formats and architectures are described in Holliger & Hudson, 2006, Nature Biotechnology 23(9):1126-1136, and Carter 2006, Nature Reviews Immunology 6:343-357 and references cited therein, all expressly incorporated by reference. In additional embodiments, antibodies are produced by enzymatic or chemical cleavage of naturally occurring antibodies.

Nanoparticles

The present disclosure is broadly directed to nanoparticles having a lipid layer disposed on the surface of a porous framework core. The porous framework core includes a porous framework material that includes compounds within the pores. As such, the particles can be considered “artificial cells” that have a membrane-like exterior and interior adapted to contain compounds such as therapeutic or diagnostic compounds of different hydrophobicity or hydrophilicity.

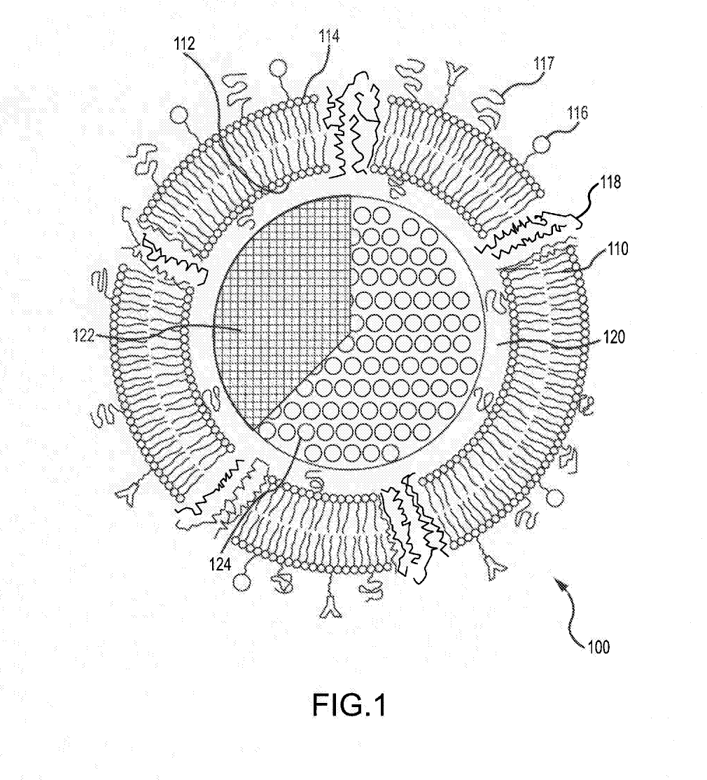

As shown in FIG. 1, in one aspect, particle 100 has a lipid bilayer 110 disposed on the surface of a porous framework core 120. The porous framework core includes a porous framework material 122, and one or more compounds 124 within the pores. The lipid bilayer 110 includes two lipid layers, an inner layer 112 and an outer layer 114. The lipid bilayer 110 may further include molecules 116, polymers 117, and proteins 118. In various embodiments, the molecules 116, polymers 117, and proteins 118 of the lipid bilayer 110 may aid in retention or targeting of the particle 100 to specific locations in a patient, the release of one or more compounds 124 from the nanoparticle 100, or fusion of the nanoparticle 100 with a target cell.

The nanoparticles as described herein are generally on the nanoscale. In general, nanoscale nanoparticles measure between 1 and 1000 nanometers in at least one measurable dimension. In various embodiments, the nanoparticles may measure greater than 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, or 240 nm in at least one measureable dimension. In other embodiments, nanoparticles may measure less than 250 nm, 240 nm, 230 nm, 220 nm, 210 nm, 200 nm, 190 nm, 180 nm, 170 nm, 160 nm, 150 nm, 140 nm, 130 nm, 120 nm, 110 nm, 100 nm, 90 nm, 80 nm, 70 nm, 60 nm, 50 nm, or 40 nm in at least one measurable dimension. Nanoparticles may have various shapes, including rods, spheres, and platelets.

The nanoparticle may have pores of various dimensions, and are said to be mesoporous (e.g. pore size in the range of 2 nm to 50 nm). In various embodiments, the pores are greater than 0.5 nm, 1 nm, 2 nm, 3 nm, 4 nm, 5 nm, 6 nm, 7 nm, 8 nm, 9 nm, 10 nm, 11 nm, 123 nm, 13 nm, 14 nm, 15 nm, 16 nm, 17 nm, 18 nm, 19 nm, 20 nm, 25 nm, 30 nm, 35 nm, 40 nm, or 45 nm. In various other embodiments, the pores may be less than 50 nm, 45 nm, 40 nm, 35 nm, 30 nm, 25 nm, 20 nm, 19 nm, 18 nm, 17 nm, 16 nm, 15 nm, 14 nm, 13 nm, 12 nm, 11 nm, 10 nm, 9 nm, 8 nm, 7 nm, 6 nm, 5 nm, 4 nm, 3 nm, 2 nm, or 1 nm. As described herein, the pore sizes may be “tunable” (i.e. selected). In various examples, this may be accomplished by changing the size of molecular assemblies that direct the pore formation, or by use of a swelling agent. By way of example and not limitation, changing the alkyl chain length of the cationic surfactant used to direct pore formation in a alumniosilicate gel/surfactant reaction, will alter the pore size (as described in Kresge C. T. et al, Nature, 359, 710 (1992), changing the surfactant used to direct pore formation from C₁₆H₃₃(CH₃)₃N⁺Cl⁻ to C₁₂H₂₅(CH₃)₃N⁺Cl⁻, resulted in the pore size changing from ˜4 nm to ˜3 nm).

Larger pore sizes may also be directed by various techniques well known in the art. In some embodiments, addition of an organic molecule such as, 1,3,5-trimethylbenzene, to the synthesis reaction may lead to enlarged pore sizes (as described in Kresge C. T. et al, (Nature, 359, 710 (1992); addition of 1,3,5,-trimethylbenzene increased pore size up to approximately 10 nm). In other embodiments, nanoparticle framework material may be synthesized in reactions with an amphiphilic block copolymer to direct pore formation as described by Zhao D. Y. et al, (Science, 279, 548, (1998)). In this embodiments, a tri-block copolymer such as, for example without limitation, Pluronic P123 (EO₂₀PO₇₀EO₂₀) may be used to direct larger pore sizes in a reaction comprising tetraethoxy-, tetramethoxy-, or tetrapropoxy-silane as suitable sources of silica.

The particles include a lipid layer-layer disposed on a porous framework core.

A. Porous Framework Core

The porous framework core of the nanoparticles described herein includes a porous framework material and a compound, such as a therapeutic or diagnostic compound.

Various materials may be used to construct the porous framework material. In some embodiments, porous framework material is a silica. Alternatively, metal oxide porous framework materials may be used, including, but not limited to, Ti, Zr, Nb, W, In, Sn, Ta, Hf, Al, Fe, and Ce. In further embodiments, non-metal oxide materials like carbon may be used. (see for example, Dong A et al., JACS, 125, 4976 (2003), Ryoo R et al, Adv Mater, 13, 677 (2001), Tian B Z et al, Nat Mater, 2, 159 (2003)). Alternative embodiments may include non-silica, non-metal porous framework materials including carbon, carbon nitride, boron nitride etc. Further, those skilled in the art will recognize that different materials may be combined in the manufacture of these particles.

In one embodiment, porous framework material comprises silica. Silica is a non-immunogenic, nontoxic, biocompatible material (M. Ferrari, Nat. Rev. Cancer 5, 161 (March, 2005), S. R. Blumen et al., Am. J. Respir. Cell Mol. Biol. 36, 333 (March, 2007)). Alternatively, non-silica porous framework material may be coated with silica.

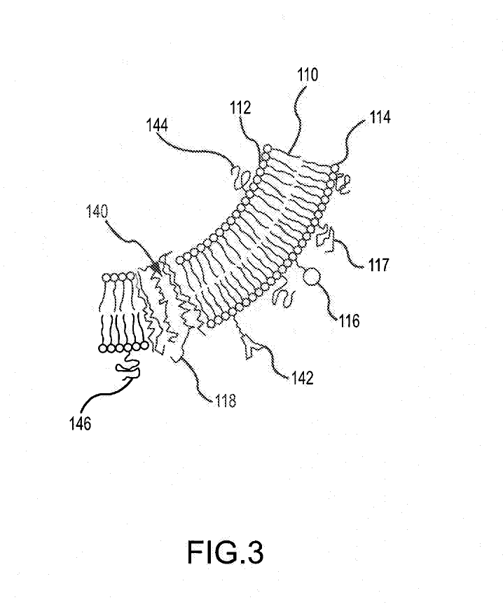

The pores of the porous framework material may have any shape known in the art. For example, as depicted in FIG. 3, the pores 130 of the porous framework material may create hexagonally shaped openings, while in other aspects the pores may be generally circular or oval (see for example, Park et al, Angew. Chem. Int. Ed. 2007, 46, 1455-1457, and Zhao et al, Science, 279, 548 (1998)). In further aspects, the pores may be randomly shaped. The pores may be uniformly arranged in a regular density (i.e. number of pores per surface area of the porous framework material), or may vary in pore density throughout the porous framework material. In other examples, the pores may extend through the particle.

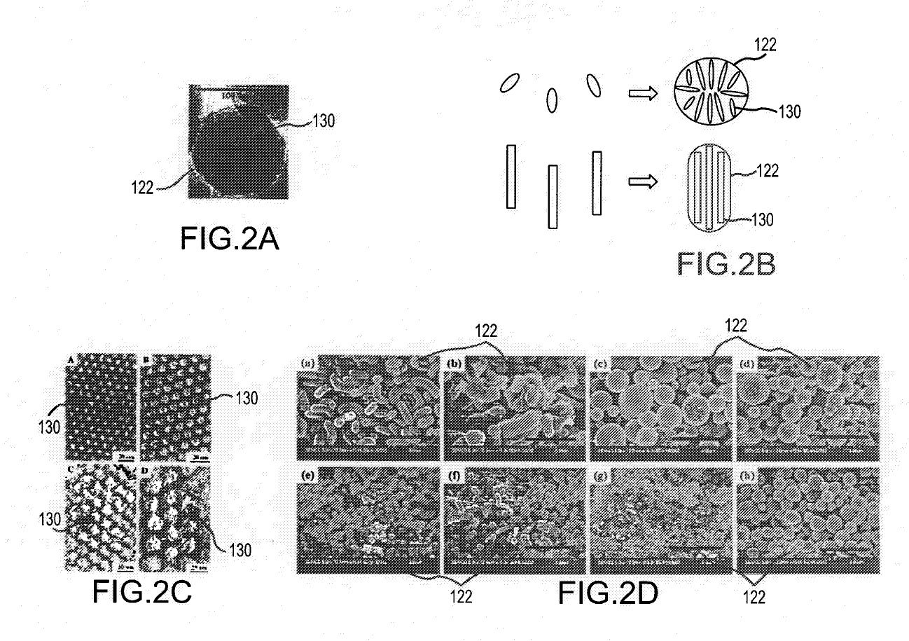

FIG. 2 depicts various structural characteristics of the porous framework material 122. The size and shape of the pores 130 as well as of the porous framework material 122 may be selected, for example as described in Trewyn et al, Adv. Funct. Mater. 17, 1225-1236 (2007), and Slowing et al, J. Am. Chem. Soc. 129, 8845 (Jul. 18, 2007)). As shown in the embodiment depicted in FIG. 3A, the porous framework material 122 is spherical with a plurality of pores 130. The porous framework material 122 has allowed the manufacture of variously shaped porous framework material as depicted in FIG. 3B where pores 130 may be aligned to create a rod-shaped porous framework material 130, or spherical porous framework cores. FIG. 3C shows porous framework material 122 with pores 130 of varying sizes (see, Id. and references therein). In some aspects the overall size of the porous framework material 122 may be between 50 and several hundred nanometers with pore sizes ranging from 2 to tens of nanometers, and the surface area may range from 700 to 1500 cm²/g, for example as described in Trewyn et al.; R. I. Nooney, et al, Chem. Mater. 14, 4721 (November, 2002); Y. Han, J. Y. Ying, Angew. Chem. Int. Ed. 44, 288 (2005), Y. F. Lu et al., Nature 398, 223 (Mar. 18, 1999), and Y. S. Lin et al., Chem. Mater. 17, 4570 (Sep. 6, 2005). Specific porous framework material 122 characteristics may be varied and selected based on the desired application, such as a delivery system for compounds or nucleic acid (See generally, Slowing et al, J. Am. Chem. Soc. 129, 8845 (Jul. 18, 2007)).

In various embodiments, pores may extend through the framework material. Further, channels may interconnect, to form a collection of interconnecting channels. Alternatively, pores may create pockets at the surface of the framework material, and may have a single opening at the surface of the framework material.

The porous framework material may be further modified depending on the intended application. For example, the porous framework material may be derivatized at the surface of the particle or alternatively the interior of the pores may be modified to add functionality, or chemical groups. Derivatization may be performed either during synthesis of the particles (“co-condensation”) or through attaching molecules to the particles after the particles are formed (“post-modification”).

In some embodiments the porous framework material may include one or more functional moieties. Each functional moiety independently may include an alkyl, aryl, hydroxyl, carboxyl, amine, amino, thio, epoxy, cyano, or halogen like that described in Hoffman F et al, Angew. Chem. Int. Ed. 45, 3216 (2006); Stein A et al, Adv Mater., 12, 1403 (2000); and Vallet-Regi M et al., Angew. Chem. Int. Ed. 46, 7548 (2007)). For example and not limitation, functional silanes may include phenyltriethoxysilane (PTES), octyltriethoxysilane (OTES), allyltrimethoxysilane (ATMS), 3-mercaptopropyltrimethoxysilane (MPTMS), 3-aminopropyltriethoxysilane (APTES), 3-(2,3-epoxypropoxy)propyltrimethoxysilane, 3-imidazolyltriethoxysilane. In one aspect, a co-condensation process, may include a functional silane (such as, without limitation, APTES that gives a —NH₂ derivalized surface) may be used with a non-functionalized silane (such as TEOS) in a molar ratio from 1:20 to 1:1 during nanoparticle growth. In a non-limiting example of a post-modification process, a 1-5% V/V functional silane solution (such as APTES that gives a —NH₂ derivalized surface) may be prepared using anhydrous solvents such as acetone or toluene. In this example, dry silica porous framework material may be added into the silane solution, and the reaction fluxed overnight under stirring and protection from nitrogen. Further in this example, the surface of the porous framework material may be washed extensively in anhydrous solvents such as acetone or toluene, and dried in vacuum oven.

Porous framework material may be modified by a variety of different molecules. For example, the particles may be coated with polyethyleneglycol (as described, for example, in Slowing et al.) or with functional silanes. Cleavable groups may be added to the functional silane, such as without limiting by example, a di-sulfide group.

Porous framework material may also be chemically modified to aid the uptake of compounds having specific properties by functionalizing the porous framework material with functional groups. For example, the porous framework material can be adapted to contain hydrophobic compounds (See generally, Vallet-Regi et al Angew. Chem. Int. Ed. 2007, 46, 7548-7558). For example, the porous framework material may be functionalized with amino groups, chloro groups, sulfo groups, and larger molecules such as benzyl-containing groups.

Surface silanization may be carried out to facilitate functionalization of the porous framework material. In various embodiments, silanization may include preparing a functional silane solution using anhydrous solvents and adding a dry porous framework material to the silane solution, followed by allowing the solution to react then washing and drying the particles. In some embodiments the functional silanes may have a generic form of R¹ₓ—Si—(OR²)₄₋ₓ, where x is 1, 2, or 3, R² is usually an alkyl-group, R¹ is an alkyl chain with a functional moiety as the end group. In some embodiments the functional moiety may be alkyl, aryl, hydroxyl, carboxyl, amine, amino, thio, epoxy, cyano, or halogen.

Stimuli-responsive molecules, polymers, or proteins can also be grafted onto the porous framework material. These molecules, polymers, or proteins would allow compounds to be released from the pores in response to physiological variations including, without wishing to limit by example, changes in temperature, pH, or ionic strength surrounding the porous framework material. Additional molecules may be found in the Merck Index, 13^{th} ed., incorporated herein by reference in its entirety.

The porous framework core includes compounds within the pores of the porous framework material. In various embodiments, the compounds may be therapeutic and diagnostic compounds. Any compound in the art may be included. Compounds may include any compound in the Merck Index, 13^{th} ed., incorporated herein by reference in its entirety.

Therapeutic agents can be, but are not limited to, steroids, analgesics, local anesthetics, antibiotic agents, chemotherapeutic agents, immunosuppressive agents, anti-inflammatory agents, antiproliferative agents, antimitotic agents, angiogenic agents, antipsychotic agents, central nervous system (CNS) agents; anticoagulants, fibrinolytic agents, growth factors, antibodies, ocular drugs, and metabolites, analogs, derivatives, fragments, and purified, isolated, recombinant and chemically synthesized versions of these species, and combinations thereof.

Representative useful therapeutic agents include, but are not limited to, tamoxifen, paclitaxel, anticancer drugs, camptothecin and its derivatives, e.g., topotecan and irinotecan, KRN 5500 (KRN), meso-tetraphenylporphine, dexamethasone, benzodiazepines, allopurinol, acetohexamide, benzthiazide, chlorpromazine, chlordiazepoxide, haloperidol, indomethacine, lorazepam, methoxsalen, methylprednisone, nifedipine, oxazepam, oxyphenbutazone, prednisone, prednisolone, pyrimethamine, phenindione, sulfisoxazole, sulfadiazine, temazepam, sulfamerazine, ellipticin, porphine derivatives for photo-dynamic therapy, and/or trioxsalen, as well as all mainstream antibiotics, including the penicillin group, fluoroquinolones, and first, second, third, and fourth generation cephalosporins. These agents are commercially available from, e.g., Merck & Co., Barr Laboratories, Avalon Pharma, and Sun Pharma, among others.

Additional classes of therapeutic agents include, but are not limited to, compounds for use in the following therapeutic areas: antihypertensives, antianxiety agents, antiarrythmia agents, anticlotting agents, anticonvulsants, blood glucose-lowering agents, decongestants, antihistamines, antitussives, antineoplastics, beta blockers, anti-inflammatories, antipsychotic agents, cognitive enhancers, anti-atherosclerotic agents, cholesterol-reducing agents, triglyceride-reducing agents, antiobesity agents, autoimmune disorder agents, anti-impotence agents, antibacterial and antifungal agents, hypnotic agents, anti-Parkinsonism agents, anti-Alzheimer's disease agents, antibiotics, anti-angiogenesis agents, anti-glaucoma agents, anti-depressants, and antiviral agents.

Each named therapeutic agent should be understood to include the nonionized form of the therapeutic agent or pharmaceutically acceptable forms of the therapeutic agent. By “pharmaceutically acceptable forms” is meant any pharmaceutically acceptable derivative or variation, including stereoisomers, stereoisomer mixtures, enantiomers, solvates, hydrates, isomorphs, polymorphs, pseudomorphs, neutral forms, salt forms and prodrug agents.

Additional exemplary therapeutic agents suitable for use in the nanoparticles include, but are not limited to, phosphodiesterase inhibitors, such as sildenafil and sildenafil citrate; HMG-CoA reductase inhibitors, such as atorvastatin, lovastatin, simvastatin, pravastatin, fluvastatin, rosuvastatin, itavastatin, nisvastatin, visastatin, atavastatin, bervastatin, compactin, dihydrocompactin, dalvastatin, fluindostatin, pitivastatin, and velostatin (also referred to as synvinolin); vasodilator agents, such amiodarone; antipsychotics, such as ziprasidone; calcium channel blockers, such as nifedipine, nicardipine, verapamil, and amlodipine; cholesteryl ester transfer protein (CETP) inhibitors; cyclooxygenase-2 inhibitors; microsomal triglyceride transfer protein (MTP) inhibitors; vascular endothelial growth factor (VEGF) receptor inhibitors; carbonic anhydrase inhibitors; and glycogen phosphorylase inhibitors. Other low-solubility therapeutic agents suitable for use in the nanoparticles are disclosed in US Published patent application 2005/0031692, herein incorporated by reference.

Therapeutic compounds may also be used to achieve a desired prophylactic result, i.e. therapeutic compounds may be used prophylactively. Typically, prophylaxis is achieved prior to or at an earlier stage of disease than that treated by a therapeutic compound. Diagnostic compounds aid in determining whether a disease state exists in a patient. Alternatively, diagnostic compounds may aid in imaging, or measuring metabolic function.

In some embodiments, the compounds may be hydrophobic or partially hydrophobic (J. Lu, et al, Small 3, 1341 (August, 2007)). Hydrophobic compounds possess non-polar characteristics and thus not readily soluble in polar environments.

The therapeutic agent may be“hydrophobic” or “poorly water soluble,” meaning that the therapeutic agent has a solubility in water (over the pH range of 6.5 to 7.5 at 25° C.) of less than 5 mg/mL. The utility of the disclosure increases as the water solubility of the therapeutic agent decreases. The therapeutic agent may have an even lower solubility in water, such as less than about 1 mg/mL, less than about 0.1 mg/mL, and even less than about 0.01 mg/mL. In general, it may be said that the therapeutic agent has a dose-to-aqueous solubility ratio greater than about 10 mL, and more typically greater than about 100 mL, where the aqueous solubility (mg/mL) is the minimum value observed in any physiologically relevant aqueous solution (i.e., solutions with pH 1-8), including USP simulated gastric and intestinal buffers, and dose is in mg. Thus, a dose-to-aqueous solubility ratio may be calculated by dividing the dose (in mg) by the aqueous solubility (in mg/mL).

In another embodiment, the therapeutic agent is a hydrophobic non-ionizable therapeutic agent. By “hydrophobic non-ionizable therapeutic agent” is meant a subclass of non-ionizable therapeutic agents that are essentially water insoluble and highly hydrophobic, and are characterized by a set of physical properties, as described herein. By “non-ionizable” is meant that the therapeutic agent has substantially no ionizable groups. By “ionizable groups” is meant functional groups that are at least about 10% ionized over at least a portion of the physiologically relevant pH range of 1 to 8. Such groups have pKa values of about 0 to 9. Thus, hydrophobic non-ionizable therapeutic agents do not have a pKa value between 0 and 9.

In other embodiments, the compounds may be or contain proteins or peptides (Slowing, II, et al, J. Am. Chem. Soc. 129, 8845 (Jul. 18, 2007)). In further embodiments, the compounds may be DNA, RNA, or other nucleic acids (S. M. Solberg, C. C. Landry, J. Phys. Chem. B 110, 15261 (Aug. 10, 2006)). Compounds may also include biologics such as, without wishing to be limited by example, vaccines, blood products, and peptides. In some embodiments, more than one type of compound may be included in the porous framework core.

The nanoparticles described herein can be used to treat diseased cells and tissues. In this regard, various diseases are amenable to treatment using the nanoparticles and methods described herein. An exemplary, nonlimiting list of diseases that can be treated with the subject nanoparticles includes breast cancer; prostate cancer; lung cancer; lymphomas; skin cancer; pancreatic cancer; colon cancer; melanoma; ovarian cancer; brain cancer; head and neck cancer; liver cancer; bladder cancer; non-small lung cancer; cervical carcinoma; leukemia; non-Hodgkins lymphoma, multiple sclerosis, neuroblastoma and glioblastoma; T and B cell mediated autoimmune diseases; inflammatory diseases; infections; hyperproliferative diseases; AIDS; degenerative conditions, cardiovascular diseases, transplant rejection, and the like. In some cases, the treated cancer cells are metastatic.

The route and/or mode of administration of a nanoparticle described herein can vary depending upon the desired results. Dosage regimens can be adjusted to provide the desired response, e.g., a therapeutic response.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. The mode of administration is left to the discretion of the practitioner.

In some instances, a nanoparticle described herein is administered locally. This is achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as sialastic membranes, or fibers. In some situations, a nanoparticle described herein is introduced into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

This disclosure also features a device for administering a nanoparticle described herein. The device can include, e.g., one or more housings for storing pharmaceutical compositions, and can be configured to deliver unit doses of a nanoparticle described herein.

Pulmonary administration can also be employed, e.g., by use of an inhaler or nebulizer, and formulation with an aerosolizing agent, or via perfusion in a fluorocarbon or synthetic pulmonary surfactant.

A nanoparticle described herein is formulated as a pharmaceutical composition that includes a suitable amount of a physiologically acceptable excipient (see, e.g., Remington's Pharmaceutical Sciences pp. 1447-1676 (Alfonso R. Gennaro, ed., 19th ed. 1995)). Such physiologically acceptable excipients can be, e.g., liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one situation, the physiologically acceptable excipients are sterile when administered to an animal. The physiologically acceptable excipient should be stable under the conditions of manufacture and storage and should be preserved against the contaminating action of microorganisms. Water is a particularly useful excipient when a nanoparticle described herein is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Other examples of suitable physiologically acceptable excipients are described in Remington's Pharmaceutical Sciences pp. 1447-1676 (Alfonso R. Gennaro, ed., 19th ed. 1995). The pharmaceutical compositions, if desired, can also contain minor amounts of wetting or emulsifying agents, or pH buffering agents.

Liquid carriers can be used in preparing solutions, suspensions, emulsions, syrups, and elixirs. A nanoparticle described herein can be suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives described herein, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. The liquid carriers can be in sterile liquid form for administration. The liquid carrier for pressurized compositions can be halogenated hydrocarbon or other pharmaceutically acceptable propellant.

In other instances, a nanoparticle described herein is formulated for intravenous administration. Compositions for intravenous administration can comprise a sterile isotonic aqueous buffer. The compositions can also include a solubilizing agent. Compositions for intravenous administration can optionally include a local anesthetic such as lignocaine to lessen pain at the site of the injection. The ingredients can be supplied either separately or mixed together in unit dosage form, for example, as a dry lyophilized powder or water-free concentrate in a hermetically sealed container such as an ampule or sachette indicating the quantity of active agent. Where a nanoparticle described herein is administered by infusion, it can be dispensed, for example, with an infusion bottle containing sterile pharmaceutical grade water or saline. Where a nanoparticle described herein is administered by injection, an ampule of sterile water for injection or saline can be provided so that the ingredients can be mixed prior to administration.

In other circumstances, a nanoparticle described herein can be administered across the surface of the body and the inner linings of the bodily passages, including epithelial and mucosal tissues. Such administrations can be carried out using a nanoparticle described herein in lotions, creams, foams, patches, suspensions, solutions, and suppositories (e.g., rectal or vaginal). In some instances, a transdermal patch can be used that contains a nanoparticle described herein and a carrier that is inert to the nanoparticle described herein, is non-toxic to the skin, and that allows delivery of the agent for systemic absorption into the blood stream via the skin. The carrier can take any number of forms such as creams or ointments, pastes, gels, or occlusive devices. The creams or ointments can be viscous liquid or semisolid emulsions of either the oil-in-water or water-in-oil type. Pastes of absorptive powders dispersed in petroleum or hydrophilic petroleum containing a nanoparticle described herein can also be used. A variety of occlusive devices can be used to release a nanoparticle described herein into the blood stream, such as a semi-permeable membrane covering a reservoir containing the nanoparticle described herein with or without a carrier, or a matrix containing the nanoparticle described herein.

A nanoparticle described herein can be administered rectally or vaginally in the form of a conventional suppository. Suppository formulations can be made using methods known to those in the art from traditional materials, including cocoa butter, with or without the addition of waxes to alter the suppository's melting point, and glycerin. Water-soluble suppository bases, such as polyethylene glycols of various molecular weights, can also be used.

The amount of a nanoparticle described herein that is effective for treating disorder or disease is determined using standard clinical techniques known to those with skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. For example, the dose of a nanoparticle described herein can each range from about 0.001 mg/kg to about 250 mg/kg of body weight per day, from about 1 mg/kg to about 250 mg/kg body weight per day, from about 1 mg/kg to about 50 mg/kg body weight per day, or from about 1 mg/kg to about 20 mg/kg of body weight per day. Equivalent dosages can be administered over various time periods including, but not limited to, about every 2 hrs, about every 6 hrs, about every 8 hrs, about every 12 hrs, about every 24 hrs, about every 36 hrs, about every 48 hrs, about every 72 hrs, about every week, about every two weeks, about every three weeks, about every month, and about every two months. The number and frequency of dosages corresponding to a completed course of therapy can be determined according to the judgment of a health-care practitioner.

In some instances, a pharmaceutical composition described herein is in unit dosage form, e.g., as a tablet, capsule, powder, solution, suspension, emulsion, granule, or suppository. In such form, the pharmaceutical composition can be subdivided into unit doses containing appropriate quantities of a nanoparticle described herein. The unit dosage form can be a packaged pharmaceutical composition, for example, packeted powders, vials, ampoules, pre-filled syringes or sachets containing liquids. The unit dosage form can be, for example, a capsule or tablet itself, or it can be the appropriate number of any such compositions in package form. Such unit dosage form can contain from about 1 mg/kg to about 250 mg/kg, and can be given in a single dose or in two or more divided doses.

Compounds in the porous framework core may have a variety of characteristics. In some aspects the compound may be hydrophobic, hydrophilic, or amphipathic. Hydrophobic compounds are generally non-polar molecules that do not readily dissolve in water. Hydrophobic compounds may be readily soluble in organic solvents. The retention of hydrophobic compounds by the porous framework material can be increased by functionalizing the porous framework material to include hydrophobic groups, as discussed here. Alternatively, the retention of hydrophilic compounds, which are generally soluble in water or other polar solvents, can be augmented by functionalizing porous framework material with hydrophilic or polar groups. Alternatively, retention of amphipathic compounds, which possess hydrophobic and hydrophilic characteristics, may be augmented by functionalizing the porous framework material with both polar and hydrophilic groups.

In some aspects, the compounds of the porous framework core may be insoluble or soluble in a given solution or solvent. In some embodiments, insoluble compounds do not dissolve readily in water or other polar solvents. In other embodiments, the compounds do not dissolve in organic solvents. In some aspects an insoluble compound may be solubilzed through mechanical methods such as mixing, or sonication where insoluble compounds are broken apart with sound waves. Solubility may also be affected by temperature, pH, and ionic strength of a given solvent.

The nanoparticles disclosed herein may be adapted to administer compounds that are ineffective by traditional routes of administration. As described above, the nanoparticles can be adapted to administer hydrophobic compounds. Alternatively, the nanoparticles can be adapted to administer toxic compounds, readily labile compounds, or compounds that have low effectiveness in the patient.

In some aspects, the nanoparticles disclosed herein provide a methods of targeting compounds to a specific location in an organism. Toxic compounds include those that may have an LD50 expressed as milligrams of compound per kilogram bodyweight at which half a tested population will die. Chemotherapeutic compounds, for example, are toxic at certain concentrations. In some aspects toxic compounds include those in the mg/kg LD50 range. In some embodiments, the compounds may have an LD50 expressed in nanograms/kg or femtograms/kg. In various embodiments, toxic compounds cannot be introduced to a subject in unmodified form, or in the absence of a carrier. However, when included in the nanoparticles described herein, a toxic compound may be targeted to a specific location in the organism prior to release.

In some aspects, the compound of the porous framework core may be labile. Labile compounds have very short half-lives when administered intravenously or orally. A half-life is the time required to reduce the effectiveness of the compound by half. In various embodiments, labile compounds may have a half-life of less than one hour, or half-lives of less than 24 hours. Labile compounds readily degrade when administered to a patient. However, when included in the nanoparticles disclosed herein, labile compounds are not readily degraded until release from the nanoparticle. Labile compounds can be targeted to a specific location or tissue in the patient, as described herein.

In various aspects, the compound of the porous framework core have low effectiveness when administered by traditional routes of administration. In various embodiments, compound effectiveness is a measure of the ability of the compound to effect a biological function at a given concentration. Effectiveness may be measured by a compound's EC50 or the concentration at which a given compound is 50% effective. For inhibitory compounds, effectiveness is measured by the IC50, the compound concentration at which 50% of biological function is inhibited. For compounds, EC50 and IC50 are presented as g/kg. Compounds with low effectiveness have an EC50 or IC50 at or below their LD50. When administered in the nanoparticles described herein, however, the effectiveness of the nanoparticle may be increased by targeting the nanoparticle to specific locations in the patient.

B. Lipid Layer

A lipid layer is disposed on the surface of the porous nanoparticles disclosed herein. For example, the lipid layer may be disposed on the nanoparticles as described in Mornet et al, Nano Lett, 5 (2), 281-285, (2005). Lipid layers may include a bilayer, a double layer of lipid molecules. In some aspects, the lipid molecules may include one or more fatty acid chains. The fatty acid chains may be saturated or unsaturated, mono or di-substituted and/or combinations thereof. The lipids may also be derivatized at one end with atoms other than carbon or hydrogen. Where the lipids are derivatized with non-carbon atoms, the derivatized end may be referred to as the “head,” and these atoms making up the “headgroup.” Where the fatty acid chain has a head, the chain is referred to as the tail. In some aspects, the tail may be hydrophobic and the head may be hydrophilic.

Lipid layers include monolayers and bilayers. The lipid layer may be a bilayer arranged such that there is a “head” face and a “tail” face. In some aspects, the two layers of the bilayer may be arranged so that the two respective “tail” faces are juxtaposed, or alternatively the two “head” faces are juxtaposed such that the outer layers of the bilayer presents the same type of face, either “head,” when the tails are juxtaposed, or visa versa. The bilayer may be made be homogeneous, i.e. it includes only one type of lipid or alternatively it may be heterogeneous, i.e. there are various lipids included in the bilayer.

In some aspects the lipid layer may include phospholipids. Phospholipids may include lipids with at least one phosphate atom in the headgroup. Phospholipids may be naturally occurring or synthetic (see generally, Phospholipids Handbook, Cevc, G., Ed., Marcel Dekker, New York, 1993). Phospholipids may be selected from, without limiting by example, dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidic acid, palmitelaidoyloleoylphosphatidylcholine, palm itelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinoleoylphosphatidic acid or derivatives thereof. These phospholipids may also be the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in these lysophosphatidyl derivatives may be palimtoyl, oleoyl, palmitoleoyl, linoleoyl myristoyl or myristoleoyl. In some embodiments, the mixture of phospholipids comprises dioleoylphosphatidylcholine and dioleoylphosphatidylserine in a ratio of from about 4 to about 1.

In further embodiments, the lipids in the layer may include headgroups that do not contain a phosphorous atom.

Lipid bilayers may be either synthetic or naturally occurring. Natural lipids may be obtained from eukaryotic or prokaryotic sources. In other embodiments, natural and synthetic lipids may be combined in a lipid bilayer.

In various aspects, lipids in the bilayer may include functionalized headgroups. Without wishing to limit by example, lipids may include a biotin molecule or fluorescein molecule at their headgroup (see Buranda, Langmuir, (2003), 19 (5), 1654-1663). Biotin molecules bond tightly to avidin molecules allowing further addition of molecules, polymers, or proteins to the lipid bilayer that are conjugated or otherwise associated with avidin.

With further reference to FIGS. 1 and 3, lipid bilayers 110 may include molecules 116, polymers 117, and proteins 118. In some embodiments the molecules 116, polymers 117, and proteins 118 may be inserted into the lipid bilayer 110 wherein removal of the molecule 116, polymer 117, or protein 118 requires a detergent or some other type of apolar or non-polar solvent. Molecules 116, polymers 117, and proteins 118 may be trans-bilayer, meaning that a portion of the molecule 116, polymer 117, and protein 118 extends beyond the inner layer 112 and the outer layer 114 of the bilayer 110. In other embodiments the molecules 116, polymers 117, and proteins 118 only extends beyond only one layer of the bilayer 110. Further embodiments include molecules 116, polymers 117, and proteins 118 that may be attached to, interact with, or associate with molecules 116, polymers 117, and proteins 118. In further aspects, the molecules 116, polymers 117, and proteins 118 may be attached to, interacts with, or binds a lipid headgroup in the lipid bilayer.

FIG. 3 further depicts molecules 116, polymers 117, and proteins 118 of the lipid bilayer 110 that may serve various functions. For example, without wishing to be limited by example, lipid bilayer 110 associated molecules 116, polymers 117, and proteins 118 may form channels 140, or may be antibodies 142.

In other aspects the molecules 116, polymers 117, and proteins 118 may aid in retaining the nanoparticle within the patient, targeting the nanoparticle, mediating molecular interactions, and/or releasing therapeutic or diagnostic compounds from the porous framework material. For example, a polymeric molecule such as PEG may be associated with the bilayer to increase the half-life of the nanoparticle within a patient (T. M. Allen, Trends Pharmacol. Sci. 15, 215 (July, 1994), G. Gregoriadis, Trends Biotechnol. 13, 527 (December, 1995)). In some aspects, lipid bilayers may include folate to help target, or direct, the nanoparticle to cancer cells which may over express the folate receptor (J. Sudimack, et al, Adv. Drug Deliv. Rev., 41 (2000) 147-162). In other embodiments, the proteins and polymers may target the nanoparticle to specific cells, tissues, and organs through selective interaction with particles, structures, or molecules associated with the target cell or tissue. In other embodiments, SNARE (soluble N-ethylmalemide-sensitive factor attachment protein receptor) proteins may be added to the lipid bilayer and may aid in fusing the lipid bilayer with the membranes of targeted cells (M. W. Smith, and M. Gumbleton, J. Drug Target. 14, 191 (May, 2006)).

Targeting agents can include any number of compounds known in the art. In certain situations, the targeting agent specifically binds to a particular biological target. Nonlimiting examples of biological targets include tumor cells, bacteria, viruses, cell surface proteins, cell surface receptors, cell surface polysaccharides, extracellular matrix proteins, intracellular proteins and intracellular nucleic acids.

The nanoparticles and methods described herein are not limited to any particular targeting agent, and a variety of targeting agents can be used. The targeting agents can be, for example, various specific ligands, such as antibodies, monoclonal antibodies and their fragments, folate, mannose, galactose and other mono-, di-, and oligosaccharides, and RGD peptide. Examples of such targeting agents include, but are not limited to, nucleic acids (e.g., RNA and DNA), polypeptides (e.g., receptor ligands, signal peptides, avidin, Protein A, and antigen binding proteins), polysaccharides, biotin, hydrophobic groups, hydrophilic groups, drugs, and any organic molecules that bind to receptors. In some instances, a nanoparticle described herein can be conjugated to one, two, or more of a variety of targeting agents. For example, when two or more targeting agents are used, the targeting agents can be similar or dissimilar. Utilization of more than one targeting agent in a particular nanoparticle can allow the targeting of multiple biological targets or can increase the affinity for a particular target.

In some instances, the targeting agents are antigen binding proteins or antibodies or binding portions thereof. Antibodies can be generated to allow for the specific targeting of antigens or immunogens (e.g., tumor, tissue, or pathogen specific antigens) on various biological targets (e.g., pathogens, tumor cells, normal tissue). Such antibodies include, but are not limited to, polyclonal antibodies; monoclonal antibodies or antigen binding fragments thereof; modified antibodies such as chimeric antibodies, reshaped antibodies, humanized antibodies, or fragments thereof (e.g., Fv, Fab′, Fab, F(ab′)2); or biosynthetic antibodies, e.g., single chain antibodies, single domain antibodies (DAB), Fvs, or single chain Fvs (scFv).

Methods of making and using polyclonal and monoclonal antibodies are well known in the art, e.g., in Harlow et al., Using Antibodies: A Laboratory Manual: Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab′, Fab, F(ab′)2 fragments); or biosynthetic antibodies (e.g., single chain antibodies, single domain antibodies (DABs), Fv, single chain Fv (scFv), and the like), are known in the art and can be found, e.g., in Zola, Monoclonal Antibodies: Preparation and Use of Monoclonal Antibodies and Engineered Antibody Derivatives, Springer Verlag (Dec. 15, 2000; 1st edition). In some instances, the antibodies recognize tumor specific epitopes (e.g., TAG-72 (Kjeldsen et al, Cancer Res., 48:2214-2220 (1988); U.S. Pat. Nos. 5,892,020; 5,892,019; and 5,512,443); human carcinoma antigen (U.S. Pat. Nos. 5,693,763; 5,545,530; and 5,808,005); TP1 and TP3 antigens from osteocarcinoma cells (U.S. Pat. No. 5,855,866); Thomsen-Friedenreich (TF) antigen from adenocarcinoma cells (U.S. Pat. No. 5,110,911); “KC-4 antigen” from human prostrate adenocarcinoma (U.S. Pat. Nos. 4,708,930 and 4,743,543); a human colorectal cancer antigen (U.S. Pat. No. 4,921,789); CA125 antigen from cystadenocarcinoma (U.S. Pat. No. 4,921,790); DF3 antigen from human breast carcinoma (U.S. Pat. Nos. 4,963,484 and 5,053,489); a human breast tumor antigen (U.S. Pat. No. 4,939,240); p97 antigen of human melanoma (U.S. Pat. No. 4,918,164); carcinoma or orosomucoid-related antigen (CORA) (U.S. Pat. No. 4,914,021); a human pulmonary carcinoma antigen that reacts with human squamous cell lung carcinoma but not with human small cell lung carcinoma (U.S. Pat. No. 4,892,935); T and Tn haptens in glycoproteins of human breast carcinoma (Springer et ah, Carbohydr. Res., 178:271-292 (1988)), MSA breast carcinoma glycoprotein (Tjandra et al, Br. J. Surg., 75:811-817 (1988)); MFGM breast carcinoma antigen (Ishida et al, Tumor Biol, 10: 12-22 (1989)); DU-PAN-2 pancreatic carcinoma antigen (Lan et al, Cancer Res., 45:305-310 (1985)); CA125 ovarian carcinoma antigen (Hanisch et ah, Carbohydr. Res., 178:29-47 (1988)); and YH206 lung carcinoma antigen (Hinoda et al, Cancer J., 42:653-658 (1988)). For example, to target breast cancer cells, the nanoparticles can be modified with folic acid, EGF, FGF, and antibodies (or antibody fragments) to the tumor-associated antigens MUC 1, cMet receptor and CD56 (NCAM).

Other antibodies may be used to recognize specific pathogens (e.g., Legionella peomophilia, Mycobacterium tuberculosis, Clostridium tetani, Hemophilus influenzae, Neisseria gonorrhoeae, Treponema pallidum, Bacillus anthracis, Vibrio cholerae, Borrelia burgdorferi, Cornebacterium diphtheria, Staphylococcus aureus, human papilloma virus, human immunodeficiency virus, rubella virus, and polio virus).

In some instances, the targeting agents include a signal peptide. These peptides can be chemically synthesized or cloned, expressed and purified using known techniques. Signal peptides can be used to target the nanoparticles described herein to a discreet region within a cell. In some situations, specific amino acid sequences are responsible for targeting the nanoparticles into cellular organelles and compartments. For example, the signal peptides can direct a nanoparticle described herein into mitochondria. In other examples, a nuclear localization signal is used.

In other instances, the targeting agent is a nucleic acid (e.g., RNA or DNA). In some examples, the nucleic acid targeting agents are designed to hybridize by base pairing to a particular nucleic acid (e.g., chromosomal DNA, mRNA, or ribosomal RNA). In other situations, the nucleic acids bind a ligand or biological target. For example, the nucleic acid can bind reverse transcriptase, Rev or Tat proteins of HIV (Tuerk et al, Gene, 137(I):33-9 (1993)); human nerve growth factor (Binkley et al, Nuc. Acids Res., 23(16):3198-205 (1995)); or vascular endothelial growth factor (Jellinek et al, Biochem., 83(34): 10450-6 (1994)). Nucleic acids that bind ligands can be identified by known methods, such as the SELEX procedure (see, e.g., U.S. Pat. Nos. 5,475,096; 5,270,163; and 5,475,096; and WO 97/38134; WO 98/33941; and WO 99/07724). The targeting agents can also be aptamers that bind to particular sequences.

The targeting agents can recognize a variety of epitopes on biological targets (e.g., pathogens, tumor cells, or normal cells). For example, in some instances, the targeting agent can be sialic acid to target HIV (Wies et al, Nature, 333:426 (1988)), influenza (White et al, Cell, 56:725 (1989)), Chlamydia (Infect. Immunol, 57:2378 (1989)), Neisseria meningitidis, Streptococcus suis, Salmonella, mumps, newcastle, reovirus, Sendai virus, and myxovirus; and 9-OAC sialic acid to target coronavirus, encephalomyelitis virus, and rotavirus; non-sialic acid glycoproteins to target cytomegalovirus (Virology, 176:337 (1990)) and measles virus (Virology, 172:386 (1989)); CD4 (Khatzman et al, Nature, 312:763 (1985)), vasoactive intestinal peptide (Sacerdote et al, J. of Neuroscience Research, 18: 102 (1987)), and peptide T (Ruff et al, FEBS Letters, 211: 17 (1987)) to target HIV; epidermal growth factor to target vaccinia (Epstein et al, Nature, 318: 663 (1985)); acetylcholine receptor to target rabies (Lentz et al, Science 215: 182 (1982)); Cd3 complement receptor to target Epstein-Barr virus (Carel et al, J. Biol. Chem., 265: 12293 (1990)); .beta.-adrenergic receptor to target reovirus (Co et al, Proc. Natl. Acad. ScL USA, 82: 1494 (1985)); ICAM-1 (Marlin et al, Nature, 344:70 (1990)), N-CAM, and myelin-associated glycoprotein MAb (Shephey et al, Proc. Natl. Acad. ScL USA, 85:7743 (1988)) to target rhinovirus; polio virus receptor to target polio virus (Mendelsohn et al, Cell, 56:855 (1989)); fibroblast growth factor receptor to target herpes virus (Kaner et al, Science, 248: 1410 (1990)); oligomannose to target Escherichia coli; and ganglioside GMI to target Neisseria meningitides.

In other instances, the targeting agent targets nanoparticles by recognizing and/or binding factors expressed by oncogenes. These can include, but are not limited to, tyrosine kinases (membrane-associated and cytoplasmic forms), such as members of the Src family; serine/threonine kinases, such as Mos; growth factor and receptors, such as platelet derived growth factor (PDDG), SMALL GTPases (G proteins), including the ras family, cyclin-dependent protein kinases (cdk), members of the myc family members, including c-myc, N-myc, and L-myc, and bcl-2 family members.

In addition, vitamins (both fat soluble and non-fat soluble vitamins) can be used as targeting agents to target biological targets (e.g., cells) that have receptors for, or otherwise take up, vitamins. For example, fat soluble vitamins (such as vitamin D and its analogs, vitamin E, Vitamin A), and water soluble vitamins (such as Vitamin C) can be used as targeting agents.

In some embodiments, antibodies or ligands may be used to aid in site-specific targeting (T. M. Allen, Nat. Rev. Cancer 2, 750 (October, 2002), Y. S. Park, Biosci. Rep. 22, 267 (April, 2002)). Antibodies and antibody fragments are as described herein.

Molecules, polymers, and proteins associated with the lipid bilayer may also aid compound release from the framework core. Without limiting by example, molecules, polymers, and proteins may aid compound release by forming channels through conformational change or aggregation. Conformational change or aggregation may be in response to changes in pH or temperature as described in C. Park, et al, Angew. Chem. Int. Ed. 46, 1455 (2007), Q. Fu et al., Adv. Mater. 15, 1262 (Aug. 5, 2003), and L. Zhang, et al, Adv. Mater. 19, 2988 (2007)).

In some aspects, molecules, polymers, and proteins may be induced to change conformation or aggregate in response to association with endosomes or phagosomes. Without limiting by example, the lipid bilayer may be associated with pH-responsive polymers that may expand and rupture an endosome or phagosome as described in Y. Hu et al., Nano Lett. 7, 3056 (2007). Further, the lipid bilayer may include molecules, polymers, and proteins such as listeriolysin-O that may form pores within the endosome or phagosome to aid release of the compound of the framework core (D. W. Schuerch, et al, Proc. Natl. Acad. Sci. U.S.A. 102, 12537 (Aug. 30, 2005)).

Molecules, polymers, and proteins may become associated with the lipid bilayer either directly or indirectly. In some embodiments the molecules, polymers, and proteins are added to the bilayer via “detergent-assisted reconstitution” process as described in J. L. Rigaud, D. Levy, Methods Enzymol. 372, 65 (2003)). In some aspects molecules, polymers, and proteins may be solubilized with a surfactant solution, such as but not limited to the examples listed in M. Le Maire, P. Champeil, J. V. Moller, Biochim. Biophys. Acta 1508, 86 (Nov. 23, 2000).

Molecules, polymers, and proteins also may be added to the lipid bilayer via spontaneous electrostatic interaction as described in H. J. Liang, G. Whited, C. Nguyen, A. Okerlund, G. D. Stucky, Nano Lett. 8, 333 (2008), and H. J. Liang, G. Whited, C. Nguyen, G. D. Stucky, Proc. Natl. Acad. Sci. U.S.A. 104, 8212 (May 15, 2007)). In this embodiment, the charge state of the extra-bilayer domain of the compound, polymer, or protein may be altered by selective binding with electrolytes prior to association with the lipid bilayer, so that these domains will interact with an oppositely charged lipid bilayer. The composition of the lipid bilayer also may be altered in order to aid association with molecules, polymers, and proteins. For example, without wishing to limit the disclosure, the charge of lipid layer may be predominantly negative by addition of lipids with negatively charged headgroups so that positively charged molecules, proteins, and polymers may associate with the lipid bilayer.

The disclosure is also directed to a pharmaceutical composition comprising the nanoparticle as described herein, and a pharmaceutically acceptable carrier. Any pharmaceutical carrier known in the art may be used.

C. Methods of Diagnosis and Treatment

In another aspect, the disclosure is directed to a method of diagnosing a disease or disorder by administering a nanoparticle to a patient in need of diagnosis of said disease or disorder. In various embodiments, targeting molecules, polymers, or proteins associated with the lipid bilayer aid in localizing the nanoparticle to the site of the disease or disorder. The diagnostic compound within the porous framework core is released and helps to treat said disease or disorder.

In further aspects, the disclosure is directed to a method of treating a disease or disorder by administering a nanoparticle to a patient in need of treatment of said disease or disorder. In various embodiments, targeting molecules, polymers, or proteins associated with the lipid bilayer aid in localizing the nanoparticle to the site of the disease or disorder. The therapeutic compound within the porous framework core is released and helps to treat said disease or disorder.

D. Kits

A nanoparticle described herein can be provided in a kit. In some instances, the kit includes (a) a container that contains a nanoparticle and, optionally (b) informational material. The informational material can be descriptive, instructional, marketing or other material that relates to the methods described herein and/or the use of the nanoparticles, e.g., for therapeutic benefit.

The informational material of the kits is not limited in its form. In some instances, the informational material can include information about production of the nanoparticle, molecular weight of the nanoparticle, concentration, date of expiration, batch or production site information, and so forth. In other situations, the informational material relates to methods of administering the nanoparticles, e.g., in a suitable amount, manner, or mode of administration (e.g., a dose, dosage form, or mode of administration described herein). The method can be a method of treating a subject having a disorder.

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about the nanoparticles therein and/or their use in the methods described herein. Of course, the informational material can also be provided in any combination of formats.

In addition to the nanoparticles, the kit can include other ingredients, such as a solvent or buffer, a stabilizer, or a preservative. The kit can also include other agents, e.g., a second or third agent, e.g., other therapeutic agents. The components can be provided in any form, e.g., liquid, dried or lyophilized form.

The components can be substantially pure (although they can be combined together or delivered separate from one another) and/or sterile. When the components are provided in a liquid solution, the liquid solution can be an aqueous solution, such as a sterile aqueous solution. When the components are provided as a dried form, reconstitution generally is by the addition of a suitable solvent. The solvent, e.g., sterile water or buffer, can optionally be provided in the kit.

The kit can include one or more containers for the nanoparticles or other agents. In some cases, the kit contains separate containers, dividers or compartments for the nanoparticles and informational material. For example, the nanoparticles can be contained in a bottle, vial, or syringe, and the informational material can be contained in a plastic sleeve or packet. In other situations, the separate elements of the kit are contained within a single, undivided container. For example, the nanoparticles can be contained in a bottle, vial or syringe that has attached thereto the informational material in the form of a label. In some cases, the kit can include a plurality (e.g., a pack) of individual containers, each containing one or more unit dosage forms (e.g., a dosage form described herein) of the nanoparticles. The containers can include a unit dosage, e.g., a unit that includes the nanoparticles. For example, the kit can include a plurality of syringes, ampules, foil packets, blister packs, or medical devices, e.g., each containing a unit dose. The containers of the kits can be air tight, waterproof (e.g., impermeable to changes in moisture or evaporation), and/or light-tight.

The kit can optionally include a device suitable for administration of the nanoparticles, e.g., a syringe or other suitable delivery device. The device can be provided pre-loaded with nanoparticles, e.g., in a unit dose, or can be empty, but suitable for loading.

Features and details of the disclosure can be more completely understood by reference to the following descriptions of detailed examples, taken in conjunction with the figures and from the appended claims. The examples described herein are intended only to illustrate aspects of the disclosure, and are not intended to limit or otherwise constrain the disclosure as described herein.

### EXAMPLES

### Example 1

### Silica-Based Porous Framework Material

Silica nanoparticles have been produced with diameters of less than 150 nm (see for example, FIG. 2). The structure directing agent for synthesis of the porous silica nanoparticle is triblock, star diblock copolymer and oligomeric surfactant templates. In some embodiments, the triblock copolymer is the Pluoronic polymer, F127. Fluorocarbon surfactants such as FC-4 (being both hydrophobic and lipophobic) was used to modulate the growth of the porous nanoparticle. The silica source is the silicon alkoxyloxide such as Tetraethyl orthosilicate (TEOS).

F127, FC-4, HCl, H2O, TEOS were mixed in a molar ratio of approximately 0.0005:0.009:0.08:220:1. F127, FC-4, HCl and H2O were first mixed into a homogeneous solution at 30° C., then TEOS was added. The solution was then stirred at 30° C. for one day. The solution was then transferred to an autoclave for condensation at 100-120° C. and 1-3 atm for one day. The solution was then centrifuged >1000 rpm and the pelleted material air dried. The product was then calcined at 550° C. for 5 h to remove surfactants.

In some embodiments, the structure directing agent used in the synthesis of porous silica framework material is a small molecule amphiphilic surfactant, such as cetyltrimethylammonium bromide (CTAB). The molar ratio of CTAB:NaOH:TEOS:H2O was approximately 0.122:0.312:1:1226. First, CTAB was dissolved in a NaOH solution at 80° C., next the TEOS was added with stirring. The reaction continued to stir for 2-5 hours at 80° C. The solution was then centrifuged >1000 rpm, and pelleted material air dried. The product was then calcined at 550° C. for 5 h to remove surfactants. The resulting product was then washed with organic solvent, such as ethanol, to remove the surfactant.

Both process, F127- or CTAB-based, the majority of the silica porous framework material resulted in porous framework material with a diameter 50-150 nm, and pore size 2-8 nm.

A pore swelling agent, such as Mesitylene, can be used to enlarge the pore size up to 5-fold. The molar ratio of Mesitylene:structure directing agent (CTAB or F127) may be as high as 35:1.

### Example 2

### Non-Silica Based Porous Framework Materials

Non-silica porous framework material may be prepared essentially as described above for silica porous nanoparticles. In other embodiments, non-silica porous framework material may be prepared by using a carbon or a silica porous framework material as hard-template as described in, Dong A et al., JACS, 125, 4976 (2003), and Ryoo et al., Adv Mater, 13, 677 (2001) respectively. In further embodiments, non-silica porous framework material may be created by mixing appropriate “acid-base pairs” as described in Tian B Z et al, (Nat Mater, 2, 159 (2003)). Examples of non-silica metals include but are not limited to; Ti, Zr, Nb, W, Sn, Ta, Hf, Al, Fe, Co, Ce, and In. Additionally, combinations of materials are also possible. A non-limiting partial list of the possible metal-oxides include TiO₂, ZrO₂, Nb₂O₅, WO₃, SnO₂, Ta₂O₅, HfO₂, Al₂O₃, SiTiO₄, Fe₂O₃, CO₃O₄, CeO₂, and In₂O₃. A person of skill in the art would recognize that the experimental design for non-silica based porous framework material will be similar to that described for silica.

### Example 3

### Production Lipid Bilayers

Lipid bilayers were produced with phospholipids and/or headgroup-modified lipid mixtures. To prepare lipid bilayers, stock solutions of phospholipids were dissolved in volatile organic solvents (such as chloroform, chloroform/methanol mixture etc.), and mixed at defined stoichiometric ratios (0%-100%) of different phospholipid components expected in the bilayer.

The mixture was dried under N₂ flow, followed by vacuum pumping overnight to remove possible trace of solvent residues. Millipore water (18.2 MΩ) was added to the dried lipid films to obtain phospholipid bilayer solutions with defined concentrations (usually 0-50 mg/ml).

Buffer solutions at certain pH are added to the dried lipid films to obtain phospholipid bilayer solutions with defined concentrations. The choice of buffers varies widely. In some embodiments include NaCl in the concentration range from about 0 to 10% with the most preferred range from about 6 to 9%, and an appropriate buffer. Buffers, such as N-2-Hydroxyethylpiperazine-N′-2-aminoethane sulfonic acid (HEPES), 3-[N ris(Hydroxymethyl)methylamino]-2-hydroxy-propane sulfonic acid (TAPSO), 3-(N-Morpholino) propane sulfonic acid (MOPS), N-Tris-(hydroxymethyl)methyl-2-aminoethanesulfonic acid (TES), 3-[N-bis(hydroxyethyl)-amino]2-hydroxypropane sulfonic acid (DIPSO), piperazine-N,N′-bis(2-hydroxypropane-sulfonic acid) (POPSO), N-Hydroxyethylpiperazine-N′-2-hydroxypropane sulfonic (HEPPSO) and Tris-(hydroxymethyl)aminomethane (TRIS) can be used. Some buffers, such as HEPES or TAPSO, can be used in the concentration range of about 20 to 80 mM.

The lipid bilayer solution was processed to clarity by incubation at 37° C. overnight followed by ultrasonic processing. The resultant solution was filtered through a 0.2 μm filter. Freshly prepared phospholipid bilayer solutions were used within one week.

### Example 4

### Self-Assembly of the Lipid Bilayer on Porous Framework Material

Disposing the lipid bilayer on the porous framework material involved a spontaneous self-assembly process between porous framework material and lipid bilayer. Porous framework material and lipid bilayer solutions were mixed at room temperature for 1-10 hour. Lipids with charged headgroups were introduced to help the conformational coating of the lipid bilayer and maintain the stability of the nanoparticles. Nanoparticle surface chemistry was changed by grafting with surfactants or silane molecules.

The self-assembly process is driven by a combination of van der Waals interactions, electrostatic interactions, and hydrophobic interactions. Porous framework material was suspended in Millipore water or buffer with controlled pH at a concentration of ˜2.5-25 mg/ml. An equal volume of liposome solution (˜2.5-25 mg/ml) was mixed with the suspension of porous framework material by vortexing a few seconds, and the mixture is let to sit at room temperature for 30 min with occasional vortexing. Excess lipid was removed by centrifugation of the mixture at 5000 rpm for 1 min and removal of the supernatant. The lipid-coated porous framework material was subsequently washed with Millipore water or buffer with controlled pH three times before storing in that solution.]

The lipid bilayer was a mixture of a phospholipid with other phospholipids that were modified with functional moieties on their headgroups. In some embodiment, the lipid bilayer is made up by anionic phospholipid such as DOPS (1,2-Dioleoyl-sn-Glycero-3-[Phospho-L-Serine]) and zwitterionic lipid DOPC (1,2-Dioleoyl-sn-Glycero-3-Phosphocholine); in some embodiment, zwitterionic lipid DOPE (1,2-Dioleoyl-sn-Glycero-3-Phosphoethanolamine) is used instead of DOPC. The modified phospholipids were covalently and non-covalently conjugated with moieties on their headgroups for targeting. The moieties included PEG polymer, ligands, or antibodies that recognize targeted cells. The selection of suitable lipids depended on the nature of the targets and the interactions with the nanoparticle surface. Lipids with charged headgroups were also used to optimize the electrostatic interactions in the system.

### Example 5

### Insertion of Proteins into a Lipid Bilayer

Proteins (e.g. membrane proteins) are associated with the lipid bilayer via a spontaneous electrostatic interaction-driven reconstitution process (H. J. Liang, G. Whited, C. Nguyen, A. Okerlund, G. D. Stucky, Nano Lett. 8, 333 (2008), H. J. Liang, G. Whited, C. Nguyen, G. D. Stucky, Proc. Natl. Acad. Sci. U.S.A. 104, 8212 (May 15, 2007)) or detergent-assisted reconstitution process (J. L. Rigaud, D. Levy, Methods Enzymol. 372, 65 (2003)). Here, the charge state of the extra-bilayer domains of membrane proteins was tuned by pH, selective binding with electrolytes, and charged amino acid interaction with the oppositely charged phospholipid bilayer. These interactions drove spontaneous insertion of proteins into the phospholipid bilayer with orientation control, while the detergent associated with the proteins was automatically removed during this process. In some embodiments, the lipid bilayer associated proteins are the SNARE proteins (soluble N-ethylmalemide-sensitive factor attachment protein receptor). SNARE proteins mediate fusion of the phospholipid bilayer to target cells.

### Example 6

### Surface Silanization of Porous Framework Material

Surface silanization was carried out on silica-based porous framework material, as follows: a 1-5% V/V functional silane solution was prepared using anhydrous solvents such as acetone or toluene. Dry silica porous framework material was added into the silane solution, and the reaction fluxed overnight under stirring and protection from nitrogen. The surface of the porous framework material was then washed extensively in anhydrous solvents such as acetone or toluene, and dried in vacuum oven.

All references disclosed herein, whether patent or non-patent, are hereby incorporated by reference as if each was included at its citation, in its entirety.

Although the present disclosure has been described with a certain degree of particularity, it is understood the disclosure has been made by way of example, and changes in detail or structure may be made without departing from the spirit of the disclosure as defined in the appended claims.

1. A nanoparticle comprising:
a porous framework core comprising a porous framework material and a compound; and
a lipid layer disposed on the surface of the porous framework core, the lipid layer comprising a transmembrane channel configured to release the compound.

2. A nanoparticle of claim 1, wherein the porous framework material is formed from one or more metal oxides.

3. A nanoparticle of claim 1, wherein the compound of the porous framework core is a therapeutic or diagnostic agent.

4. A nanoparticle of claim 1, wherein polymers, and proteins are associated with the lipid layer.

5. A nanoparticle of claim 1, wherein the porous framework material is formed from non-metal oxide.

6. A method of diagnosing a disease or disorder comprising:
administering a nanoparticle of claim 1 to a patient in need of diagnosing of said disease or disorder.

7. A method of treating a disease or disorder comprising:
administering a nanoparticle of claim 1 to a patient in need of treatment of said disease or disorder.

8. A pharmaceutical composition comprising a nanoparticle as defined in claim 1, and a pharmaceutically acceptable vehicle.

9. A nanoparticle of claim 1, wherein the lipid layer further comprises a polymer.
